(19) United States
(12) Patent Application Publication
(10) Pub. No.: US 2016/0262729 A1
(43) Pub. Date: Sep. 15, 2016

(54) SYSTEMS AND METHODS OF REDUCING ULTRASONIC SPECKLE USING HARMONIC COMPOUNDING

(71) Applicant: EDAN INSTRUMENTS, INC., Shenzhen (CN)

(72) Inventors: Seshadri Srinivasan, Sunnyvale, CA (US); Edward A. Gardner, Sunnyvale, CA (US); Sean Murphy, Sunnyvale, CA (US)

(73) Assignee: EDAN INSTRUMENTS, INC., Shenzhen, (CN)

(21) Appl. No.: 15/066,854

(22) Filed: Mar. 10, 2016

Related U.S. Application Data
(60) Provisional application No. 62/131,673, filed on Mar. 11, 2015.

(51) Int. Cl. A61B 8/08; A61B 8/14; G01S 7/52

(52) U.S. Cl. CPC A61B 8/5269; G01S 7/52003; G01S 7/52085; G01S 7/52077; A61B 8/145; A61B 8/5246

(57) ABSTRACT

Systems and methods provided herein relate to an image processing system. The image processing system may include a beamformer module structured to receive channel data from each of at least three firings; and, a synthesis module communicably coupled to the beamformer module, the synthesis module may be structured to: combine channel data corresponding to two inverted firings to isolate a harmonic component; combine channel data from one of the two inverted firings with channel data from a third firing to isolate a fundamental component; and, combine the fundamental component with the harmonic component incoherently.

### CROSS-REFERENCE TO RELATED APPLICATIONS

This application claims the benefit of U.S. Provisional Patent Application No. 62/131,673, filed Mar. 11, 2015, which is incorporated herein by reference in its entirety.

### FIELD

The present disclosure relates to ultrasound imaging and, more particularly, to a method and system for improving ultrasound image quality by reducing speckle.

### BACKGROUND

Ultrasound imaging is an important and attractive tool for a wide variety of applications (e.g., diagnostic medical imaging, non-diagnostic medical imaging, etc.). However, the quality of ultrasound images is usually degraded by coherent wave interference, known as speckle, which shows up as small-scale brightness fluctuations or mottling superimposed on parts of the image. Compounding is a speckle-reduction and contrast-enhancing technique. Beneficially, compounding results in an increase in the signal-to-noise ratio of the image, which improves the image quality (e.g., resolution) of that image. Compounding techniques include spatial compounding and frequency compounding. Compared to spatial compounding, frequency compounding is more robust against tissue motion because sequential vectors rather than frames are summed together for compounding. In frequency compounding, images with different characteristics are summed incoherently. The drawback of frequency compounding is resolution degradation.

Examples of high resolution frequency compounding methods include wide-band frequency compounding and two-firing harmonic frequency compounding. A two-firing harmonic frequency compounding method is used in the EDAN U50 portable color Doppler diagnostic system, which is illustrated in FIG. 1. As shown in FIG. 1, a first firing 102 and a second firing 104 are used for each harmonic imaging line in the harmonic frequency compounding system 100. The second firing 104 is the inverse of the first firing 102 (i.e., second firing 104 and first firing 102 have same amplitude a(t) and opposite phase +a(t) and −a(t)). The channel data from each of the first and second firings 102 and 104 are amplified, digitized and combined coherently in a beamformer 106 that is configured to be identical for the first firing 102 and the second firing 104. Different gains may be used for signals from different transducer elements in order to control the aperture and apply an apodization function.

Harmonic signals 108 in the system 100 are isolated by summing the beam from the first firing 102 with the beam from the second firing 104. The summation cancels out the linear signal. The summed signal is then provided to a depth-dependent, band-pass filter 112 that is configured to pass the harmonic frequency (approximately twice the transmission frequency of the fundamental signal) while rejecting other frequencies. The depth-dependent, band-pass filter 112 is modified as a function of depth so that the filter adjusts to the reduction in signal frequency caused by attenuation. Following filtration, the signal is envelope detected 116 using a Hilbert filter (to produce a phase shift).

The fundamental signal 110 is isolated from the harmonic signal by taking the difference between the first beam from the first firing 102 (stored in a buffer) and the second beam from the second firing 104. Because the beam transmissions through the first and second firings 102 and 104 are inverted, subtracting the received signals cancels the non-linear signals as well as improves the signal-to-noise ratio of the beam due to averaging. The resulted signal from subtraction is provided to a depth-dependent, band-pass filter 114 that is configured to pass the fundamental signal while rejecting other frequencies. Analogous to the filter for isolating the harmonic component, this filter is depth dependent to adjust for attenuation. Following filtration, the signal is envelope detected using a Hilbert filter to produce a phase shift.

The detected harmonic and fundamental signals are weighted 122 using depth-dependent gain elements 118 and 120, respectively. For shallow depths, the gain elements 118 and 120 are set to emphasize the harmonic signal while at deep depths, the fundamental signal is given more weight. This allows the image to benefit from an increased resolution and a reduced clutter from the harmonic signal near the transducer and an increased signal strength and a reduced noise from the fundamental signal at deep depths. After a weighted combination 122 of the detected signals, further processing 124 is done on the combined signal to create an image. The two-firing harmonic frequency compounding method is used to reduce image speckles. However, there is still a need for improved systems and methods for reducing speckle in ultrasound images.

### SUMMARY

One embodiment relates to an ultrasound machine. The ultrasound machine includes an image acquisition device structured to acquire image data corresponding to an object of interest, wherein the image data includes channel data corresponding to each of a plurality of at least three firings from the image acquisition device, and wherein the image data includes a fundamental component and a harmonic component. The ultrasound machine also includes an image processing system communicably coupled to the image acquisition device, the image processing system structured to isolate the fundamental component from the harmonic component by summing channel data from a set of firings in the at least three firings and subsequently combining the isolated fundamental and harmonic components. The ultrasound machine further includes an image output device structured to provide an ultrasound image from the combined harmonic and fundamental components.

Another embodiment relates to an image processing system. The image processing system includes a beamformer module structured to receive channel data from each of at least three firings; and, a synthesis module communicably coupled to the beamformer module, the synthesis module structured to: combine channel data corresponding to two inverted firings to isolate a harmonic component; combine channel data from one of the two inverted firings with channel data from a third firing to isolate a fundamental component; and combine the fundamental component with the harmonic component incoherently.

Still another embodiment relates to method for reducing speckle in an ultrasound image. According to one embodiment, the method includes: receiving, by an image processing system, channel data specific to each of at least three firings from an image acquisition device; combining, by the image processing system, channel data from two inverted firings to isolate a harmonic component; combining, by the image processing system, channel data from one of the two inverted firings with channel data from a third firing to isolate a fundamental component; log compressing, by the image processing system, each of the isolated harmonic and fundamental components separately; and combining the log compressed isolated harmonic and fundamental components to form an image.

### BRIEF DESCRIPTION OF THE FIGURES

FIG. 1 is a schematic image of a two-firing harmonic frequency compounding method, according to one embodiment.

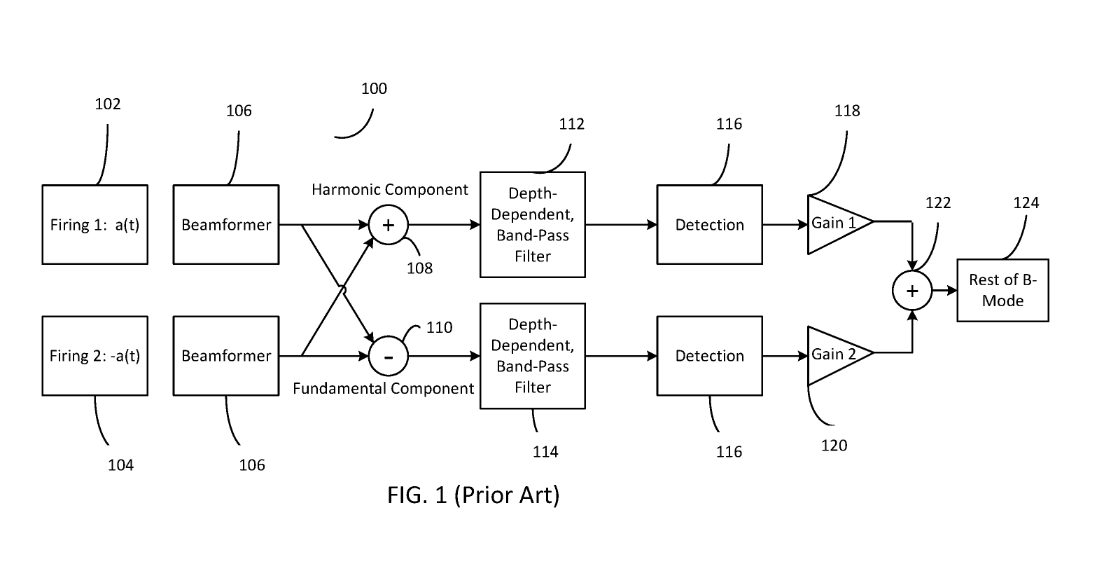

FIG. 2 is a schematic diagram of an imaging system, according to one embodiment.

FIG. 3 is a flowchart of a harmonic frequency compounding system using a four-firing configuration, according to one embodiment.

FIG. 4 is a flowchart of a harmonic frequency compounding system using a three-firing configuration, according to one embodiment.

### DETAILED DESCRIPTION

Harmonic imaging and conventional imaging are techniques used in ultrasonography. Compared to conventional imaging, harmonic imaging provides images with better quality, but limited depth. In general, a conventional ultrasound image is formed by sending out a sound pulse (i.e., a firing) to structures in the body and listening (i.e., receiving) for the transmitted pulse to echo off of one or more various structures. A harmonic image is formed by sending out a sound pulse to structures (e.g., tissue, bones, etc.) in the body, receiving the transmitted sound pulse that echoes off of the structures, and also receiving a harmonic pulse (e.g., twice the transmission frequency) generated by the structures. Therefore, the signal returned by the structures includes not only the transmitted frequency (i.e., the “fundamental” frequency), but also signals of other frequencies, most notably, the “harmonic” frequency, which is twice the fundamental frequency. Because of the differences in frequencies, different characteristics are attributable to each frequency (i.e., the fundamental frequency is able to penetrate deeper depths than the weaker harmonic frequency), where those characteristics may be leveraged by personnel to obtain relatively more detailed images depending on the object of interest (e.g., a technician may focus on a higher frequency generated image when the object is at a deeper depth within the body).

The system and method of the present disclosure is structured to reduce speckle noise without sacrificing resolution. Compared to other image compounding system and method, the present disclosure is relatively more robust against tissue motion because sequential vectors rather than frames are summed together for compounding. As described more fully herein, the method and system of the present disclosure is implemented by transmitting two or more firings, combining the two or more firings coherently to extract the harmonic and fundamental components, filtering the harmonic and fundamental components at baseband, detecting the filtered harmonic and fundamental components, applying log compression on both of the harmonic and fundamental signals and combining the compressed signals to form a compounded harmonic image. Unlike other frequency compounding systems, according to the present disclosure both the fundamental and harmonic components are created through a weighting combination of firings with different frequencies in order to extract and emphasize the signals with the frequencies of interest (e.g., firings with higher frequency for creating fundamental images) and deduct the undesired frequency signals. According to one embodiment, both the harmonic and fundamental components of the firing are processed at baseband in order to obtain a relatively greater rejection of out-of-band signals. Following the baseband processing, the processed harmonic and fundamental components are compounded (e.g., summed with gains) to create images that have relatively lower amounts of speckle to achieve higher quality images (i.e., higher resolution and contrast) relative to conventional systems. Accordingly, the generated high quality images allow users (e.g., radiologists, ultrasonography technicians, etc.) to observe a relatively greater amount of details of the targeted objects, which functions to improve the accuracy capabilities of ultrasound imaging systems.

Before turning to the Figures, which illustrate the exemplary embodiments in detail, it should be understood that the present application is not limited to the details or methodology set forth in the description or illustrated in the figures. It should also be understood that the terminology is for the purpose of description only and should not be regarded as limiting. For illustrative purposes, imaging systems using four-firing and three-firing harmonic frequency processes are shown according to various example embodiments herein.

Referring to the Figures generally, a system and method for smoothing the speckle pattern and increasing contrast resolution in ultrasound images is shown according to various embodiments herein. While the present disclosure is largely explained in regard to B-mode imaging, it should be understood that the systems and methods described herein are widely applicable. For example, the systems and methods described herein may be used with multiple other imaging modes, such as B-mode, Doppler mode (e.g., Color Doppler, Pulsed wave (PW) Doppler, etc.), Contrast, Elastography, Photoacoustics, Shear wave, Acoustic radiation force imaging mode, etc.

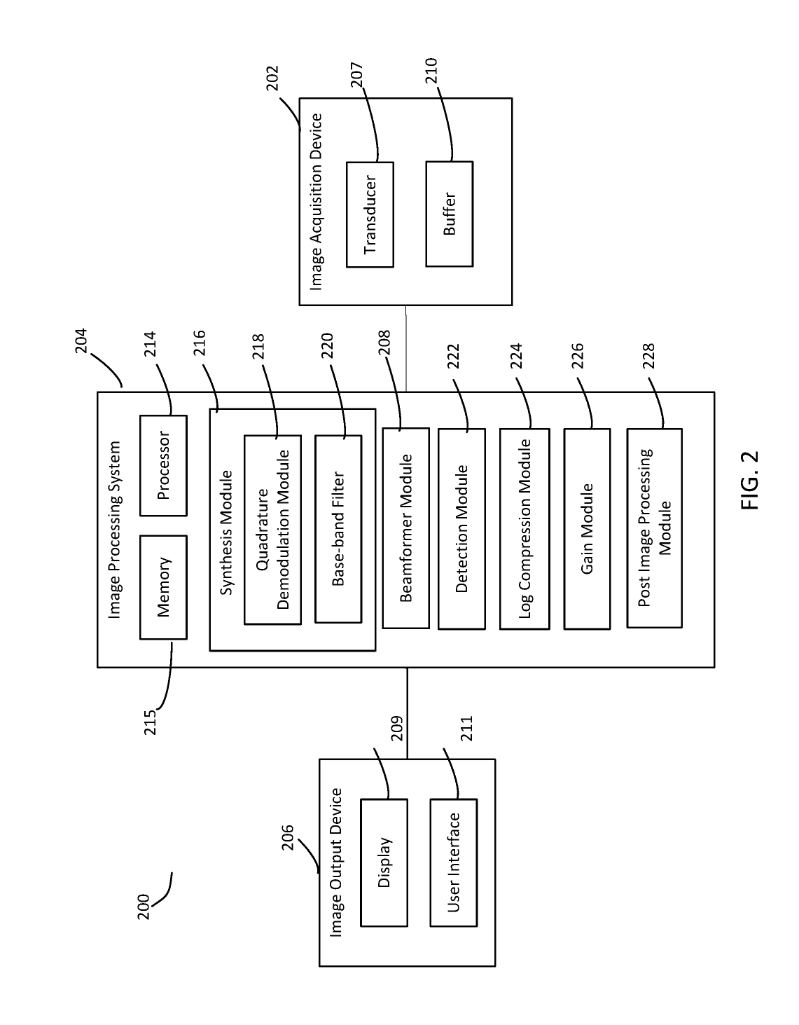

Referring now to FIG. 2, an imaging system is shown according to one embodiment. The imaging system 200 is structured to create any type of image used in ultrasonography systems. As mentioned above, according to one embodiment, the image created is a B-mode image. A B-mode image refers to a two-dimensional cross-section-image. According to other embodiments, the image is based on any type of ultrasonography system mode including, but not limited to, A-mode, C-mode, Doppler mode (e.g., Pulse Wave Doppler, Color Doppler, etc.), M-mode, harmonic mode, Acoustic radiation force imaging mode, etc. Based on the image mode utilized, the image may depict movement (e.g., a heart pulse), be in a three-dimensional space, depict color, and show various other image characteristics. Selection and application of the image mode may be based on the object to be imaged. For example, a relatively simple calf-muscle image may only need a B-mode while depiction of the organs within a chest cavity may require Doppler mode imaging. Selection and implementation of the imaging mode is therefore highly configurable and may vary based on the application.

As shown, the imaging system 200 includes an image processing system 204 communicably coupled to an image acquisition device 202 and an image output device 206. Communication between and among the components of FIG. 2 may be via any number of wired or wireless connections. For example, a wired connection may include a serial cable, a fiber optical cable, a CAT5 cable, or any other form of wired connection. In comparison, a wireless connection may include the Internet, Wi-Fi, cellular, radio, etc. In one embodiment, a controller area network (CAN) bus provides the exchange of signals, information, and/or data. The CAN bus includes any number of wired and wireless connections.

The image acquisition device 202 is structured as any type of image acquisition device utilized in ultrasonography systems. For example, the image acquisition device 202 may include, but is not limited to, an ultrasound transducer 207. The ultrasound transducer 207 may be configured as at least one of a probing (e.g., structured to be received in an opening or orifice of a patient and inserted inside the patient), a non-probing type transducer (e.g., structured to be passed over a surface of the body of a patient), or a combination of probing and non-probing type transducer. In some embodiments, the transducer 207 may be a combination of multiple transducers. In other embodiments, the transducer 207 may have multiple elements with different configurations. The transducer 207 is structured to generate and transmit a firing towards an object of interest in order to obtain image data regarding the object of interest. In one embodiment, the firing is structured as a sound wave. In this configuration, the transducer 207 is structured to convert high voltage pulses into sound waves that travel into the object of interest during transmission. In operation, the sound waves reflect off of one or more objects. The transducer 207 is structured to receive at least some of those reflections or echoes. Accordingly, each firing corresponds with specific channel data. The channel data includes the amplitude, frequency, and any other characteristic information regarding the particular firing. Image data refers to the totality of all the channel data.

The image acquisition device 202 is also shown to include a buffer 210. The buffer 210 is structured to store beams generated from transducer 207. According to one embodiment, a first firing created from the transducer 207 may be stored in the buffer 210 until a second firing is created from the transducer 207. According to another embodiment, a first firing and a third firing may be stored in the buffer until a second and/or a fourth firing are created so that harmonic and fundamental signals may be extracted from all four beams.

The image output device 206 is structured to provide the created images (e.g., to a user, radiologist, technician, other personnel, etc.). Accordingly, the image output device 206 may include, but is not limited to, a display device 209 which may be a monitor, a display screen on a computing device (e.g., a phone, tablet, etc.), a printer, a combination of these, etc. In some embodiments, the image output device 206 may include a user interface 211 configured to link to an image processing module to post-process the provided images. For example, the provided images may be adjusted with colors, contrasts, and/or focus areas through the user interface 211.

While shown as included in the image processing system 204, in some embodiments, the buffer 210 may be excluded from in the image processing system 204 (e.g., a part of the image acquisition device). Thus, the imaging system 200 may have different layouts of the devices and modules other than that illustrated in FIG. 2. All such variations are intended to fall within the spirit and scope of the present disclosure.

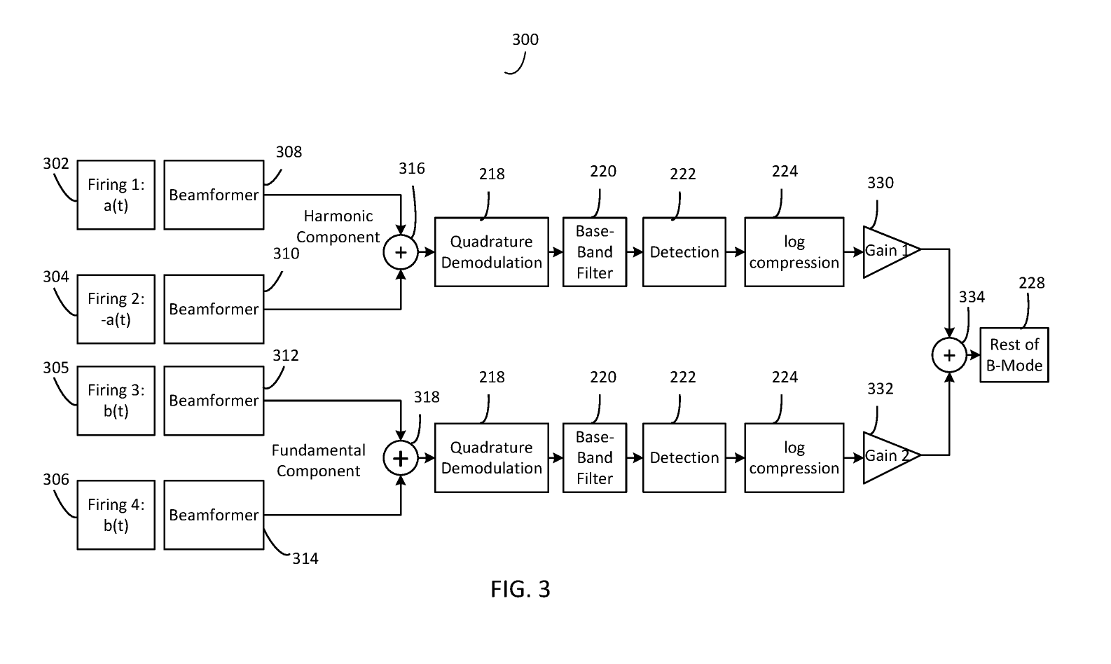

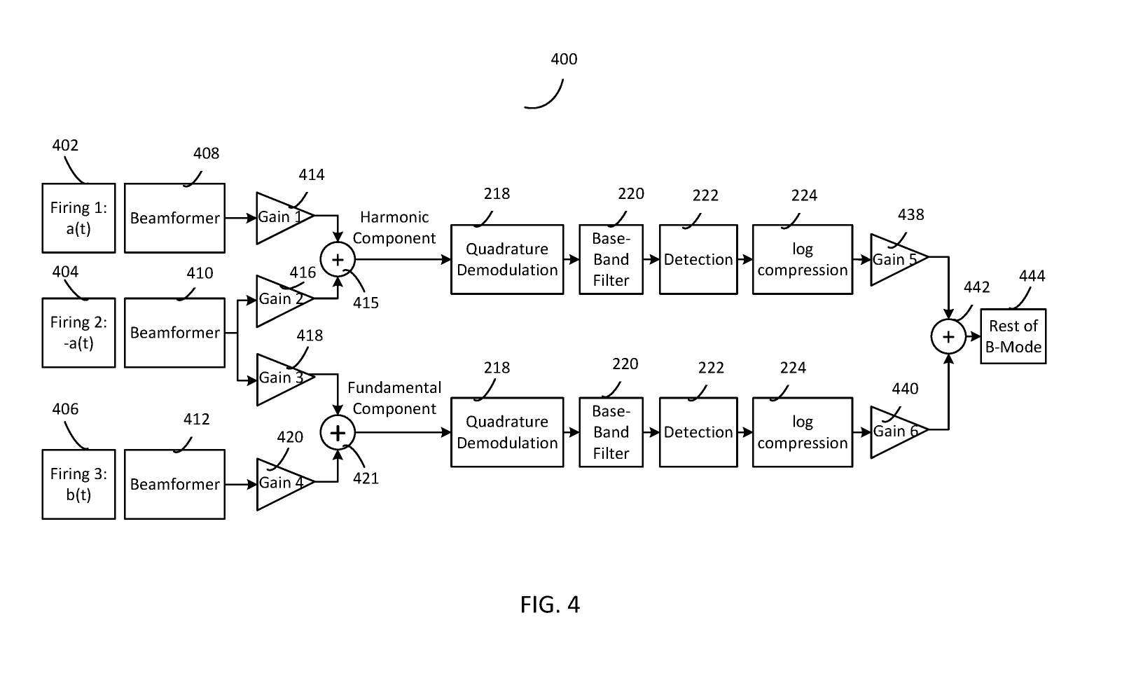

The image processing system 204 is structured to receive the beams generated from the firings to generate an ultrasound image(s). The image processing system 204 is structured to apply harmonic frequency compounding to reduce speckles and create high resolution and high quality images. Two example flow diagrams of harmonic frequency compounding systems are shown in regard to FIG. 3 and FIG. 4 herein. After compounding, the obtained high resolution images may be provided to the image output device 206 for examination by a technician (e.g., radiology expert).

An example structure of the image processing system 204 is shown in FIG. 2. The image processing system 204 includes a memory 215 and a processor 214. The processor 214 may be implemented as a general-purpose processor, and application specific integrated circuit (ASIC), one or more field programmable gate arrays (FPGAs), a digital signal processor (DSP), a group of processing components that may be spread out over a geographic area or otherwise separated, or other suitable electronic processing components. The one or more memory devices 215 (e.g., RAM, ROM, Flash Memory, hard disk storage, etc.) may store data and/or computer code for facilitating the various processes described herein. Thus, the one or more memory devices 215 may be communicably connected to the processor 214 and provide computer code or instructions to the processor 214 for executing the processes described in regard to the image processing system 204 herein. Moreover, the one or more memory devices 215 may be or include tangible, non-transient volatile memory or non-volatile memory. Accordingly, the one or more memory devices 215 may include database components, object code components, script components, or any other type of information structure for supporting the various activities and information structures described herein. As shown, imaging system 200 includes multiple modules. According to one embodiment, the modules in the system 200 may be structured as digital/machine readable programing codes. According to another embodiment, the modules in the system 200 may be structured as hardware devices (e.g., a hardware circuit, gate arrays, logic chips, transistors, resistors, etc.). Still according to another embodiment, the modules in the system 200 may be a combination of machine-readable media and hardware devices.

As shown, the image processing system 204 includes a synthesis module 216, a beamformer module 208, a detection module 222, a log compression module 224, a gain module 226, and a post image processing module 228. The synthesis module 216 is structured to isolate signals of interest. In one embodiment, the synthesis module 216 may be configured to filter out harmonic signals from the received signals. In another embodiment, the synthesis module 216 may be configured to filter out the fundamental signals from the received signals. In an alternate embodiment, the synthesis module 216 may be structured to isolate any other frequency of interest in the beam (i.e., different from either the harmonic or the fundamental frequencies).

As shown a beamformer module 208 is configured to receive the firings, which correspond to specific channel data, and form beams. The processing may include amplifying, digitizing, and coherently combining the firings within a predefined angle. In some embodiments, beamformer module 208 may be configured differently according to each firing (e.g. adjusting beam angles, scan line time intervals). According to one embodiment, the beamformer module 208 may be structured as one or more algorithms, processes, formulas, etc. Accordingly, the beamformer module 208 may be implemented in machine-readable media. In other embodiments, the beamformer module 208 may include one or more hardware components (e.g., application-specific integrated circuit (ASIC), field-programmable gate array (FPGA), digital signal processors (DSP) or a combination of these, etc. In still other embodiments, the beamformer module 208 may be a combination of multiple beamformers and each beamformer may have a specific configuration according to each firing.

As shown, the synthesis module 216 includes a quadrature demodulation module 218 and a base-band filter 220. The quadrature demodulation module 218 is configured to demodulate the received signals (i.e., harmonic signals and/or fundamental signals on a radio frequency band) to baseband signals. The baseband signals may be used to generate a relatively clear image (e.g., to depict lesions). According to one embodiment, the quadrature demodulation module 218 may be a dynamic demodulator to account for the requirements of penetration depth and signal-to-noise ratio (SNR) and reduce processing time. Accordingly, in some embodiments, the quadrature demodulation module 218 may be configured to down mix the received signals on a radio frequency (RF) band with cosine and sine values to obtain in-phase components (I) and quadrature components (Q). The Euclidean sum, \sqrt{I²+Q²}, is the magnitude of the signal while the phase is represented as arctan

$$( \frac{Q}{I} ) .$$

According to one embodiment, the quadrature demodulation module 218 varies the down-mixing along the ultrasound penetration depth to account for the change in the signals caused by depth-dependent attenuation of tissues.

The base-band filter 220 is configured to remove signals with uninterested frequencies from the images. The uninterested frequencies may be predefined by a user of the imaging system 200. For example, the base-band filter 220 may be structured to remove all signals in a non-harmonic frequency band (e.g., the fundamental signals) from the received combined signals in order to obtain harmonic signals. According to one embodiment, the baseband filter 220 may be structured as a low-pass filter to isolate the baseband signals, such that the in-phase and quadrature components from quadrature demodulation module 218 may pass through the baseband filter 220. In one embodiment, the baseband filter 220 is depth-dependent to account for the change of bandwidth caused by the depth-dependent attenuation. In another embodiment, the baseband filter 220 is a dynamic filter to account for the requirements of penetration depth and signal-noise-ration (SNR).

The detection module 222 is configured to detect the peaks of filtered signals. The envelope of the detected signals is used for compounding images. In one embodiment, the detection module 222 may be structured as a Hilbert filter. The Hilbert filter may be configured to produce a phase shifted signal based on the input signal and summing the square of the original input signal and the phase shifted signal (i.e., obtain the amplitude of combination of original and phase shifted signals). In another embodiment, the detection module 222 may be structured as a complex rotator. The complex rotator may be further configured to detect the peak frequency of the filtered baseband signals. The detected peak frequency may be used as the center frequency for the rotator. The amplitude of the complex signal (i.e., summing the square of the in-phase and quadrature components) may be used as detected signal.

The log compression module 224 is structured to reduce the dynamic range of the baseband signals using the peak frequency values from detection module 222 for efficient display. The log compression module 224 may be applied to the signals from the detection module 222 before the compounding in order to provide a relatively greater compounding effect. In some embodiments, the log compression module 224 may include parameters configured to adjust brightness of images.

The gain module 226 is structured to weight the signals (i.e., channel data). In some embodiments, gain module 226 may be configured to weight the channel data to emphasize signals with an interested or selected frequency. In other embodiments, gain module 226 is structured to weight signals from different transducers or different transducer elements in order to control the aperture (i.e., the width of the firing). In some embodiments, gain module 226 may also be configured to apply an apodization function on the signals to suppress signal sidelobes that could cause images being placed in the wrong location on the displayed image (e.g., displayed as bright and rounded lines). The gain module 226 may include multiple gain components. Each gain component may be structured to control the weight of relative signal. In some embodiments, each of the gain components in the gain module 226 may include dynamic gains and independent from each other. In other embodiments, some gain components may be relative to each other. For example, a second beam and a third beam may use the same gain value to weight for a fundamental signal.

The post image processing module 228 is structured to process the images before displaying images to users in order to further reduce speckle and increase image quality. Accordingly, the post image processing module 228 may include, but is not limited to, spatial compounding processes, digital scan conversion processes, additional speckle reduction processes, etc. In some embodiments, the post image processing module 228 may be linked to the user interface in the image output device 206 to post process the provided images as commanded by the users.

Referring now to FIG. 3, an image processing system 300 that utilizes a four-firing harmonic frequency compounding process is illustrated in schematic form, according to one embodiment. As shown, four firings 302, 304, 305, 306 are provided to beamformers 308, 310, 312, and 314. Beamformers 308, 310, 312, and 314 may have the same function and structure as described above in regard to the beamformer module 208. In some embodiments, firings 302, 304, 306, 306 may be weighted through gain module 226. Firing 302 and firing 304 are structured to be inverse relative to each other, i.e., firing 302 and firing 304 may have the same amplitude a(t) and opposite phases a(t) and −a(t). In one embodiment, firings 305 and 306 may be identical or substantially identical, but different from firings 302 and 304 (e.g., different frequencies). In another embodiment firing 305 and firing 306 may be different from each other (e.g., not identical) and from firings 302 and 304. Accordingly, in some embodiments, firings 305 and 306 may have higher transmitting frequencies compared to firings 302 and 304.

In one embodiment, Golay codes may be used for firings 305 and 306. In other embodiments, Golay codes may be used for any of the firing described herein. The Golay codes may include any type of Golay code, such as binary Golay code, extended Binary Golay code, perfect binary Golay code, etc. In still further embodiments, any other type of error correcting code may be used for firings (e.g., forward error correction, etc.). The utilization of error-correcting codes (e.g., Golay codes) may be beneficial to the detection and correction of errors within the firing. All such variations are intended to fall within the spirit and scope of the present disclosure.

Each firing is structured to obtain channel data to indicate that the acquired data is specific to a particular firing. The channel data is received by the transducer and provided to beamformers 308, 310, 312, and 314. As shown, there is one beamformer for each firing. Accordingly, each beamformer receives channel data specific to one of the first, second, third, and fourth firings. The beamformers 308, 310, 312, and 314 are structured to amplify, digitize, and coherently combine the received channel data. In some embodiments, a single beamformer may be used, but adapted differently for each firing.

The beams generated from beamformers 308 and 310 are summed together at summation element 316 to isolate the harmonic signals. The summation element 316 cancels out the linear signal components because the linear signal components in firing 302 and firing 304 are inverted. The beams generated from beamformers 312 and 314 are summed coherently at summation element 318 to reduce random noise. As shown, both the harmonic and fundamental components are demodulated into baseband through quadrature demodulation module 218 with different demodulation frequencies. The baseband harmonic and fundamental components are filtered through baseband filter module 220 to further remove unwanted frequencies. The filtered baseband harmonic and fundamental components are used for detection module 222 to generate detected harmonic and fundamental signals. The detected harmonic and fundamental components are compressed through log-compression module 224. The compressed detected signals are then combined together with a weighting function applied through gains 330 and 332 to form a compounded image. Gains 330 and 332 may be gain components in the gain module 226. In one embodiment, the weighting may emphasize the harmonic signal at shallow depths and the fundamental signal at deep depths through gain 330 and 332. For example, at shallow depths, the gain 330 for the harmonic signal may be larger than gain 332 for the fundamental signal and at deep depths, the gain 330 may be smaller than the gain 332 for the fundamental signal. In some embodiments, the gains are programmable to adjust with depth. For example, at some shallow depths, gain 332 may be larger than gain 330 (i.e., weighting may favor the fundamental in certain shallow depth as well). In some other embodiments, the gains 330 and 332 may have same value.

After forming a compounded image by combining the compressed harmonic and fundamental signals through weighting 334, the compounded image is provided to the post processing module 228.

Referring now to FIG. 4, an image processing system 400 that utilizes a three-firing harmonic frequency compounding process is illustrated in schematic form. Similar to the four-firing method and system, inverse firings 402 and 404 are used to extract the harmonic signals. Different from the four-firing configuration, a different firing 406 may be used to extract the fundamental signals. In one embodiment, firing 406 may have a different transmission pattern compared to the firings 402 and 404. Further, gain elements 414, 416, 418, and 420 are used in system 400 and configured to change the relative weighting of different firings. In one embodiment, gain elements 414 and 416 may be configured identically or substantially identical to extract the harmonic components from the combined signals at the summation element 415. In some embodiments, gain element 420 may be configured higher than gain element 418 in order to reduce the interference from the coherent summation of unrelated firings 410 and 412. The weighted firings 410 and 412 are summed to extract the fundamental signals at summation element 421. The extracted fundamental and harmonic signals are processed through demodulation 218, filter 220, detection 222, log compression 224, gains 330 and 332 (e.g., gains 438 and 440), summation element 332 (e.g., summation element 442), and post processing 228 (e.g., post processing 444) in a similar manner to the four-firing configuration. In this regard and while similar reference numbers are used to denote similar components, different reference numbers are used for gains 438 and 440, summation element 442, and post processing 444 relative gains 330 and 332, summation element 334, and post processing 228, respectively, for clarity. However, in one embodiment, these elements may be configured the same or substantially the same in each system 300, 400, such that the use of different reference numbers is not intended to necessarily denote different components.

In conventional frequency compounding system, the harmonic signal components are usually emphasized at shallow depth and fundamental signal components at deep depth. Compared to the conventional system, the present disclosure may further reduce speckles at both shallow and deep depth by improving quality of the fundamental signal components. According to the present disclosure, the fundamental and harmonic signal components are generated using different sets of firings, such as, firings 305 and 306 in the four-firing system, firings 406 and 404 in the three-firing system. The present system provides a relatively better rejection of out-of-band components by demodulating both of harmonic and fundamental signal components prior to compounding. In addition, the present system improves the compounding effect by applying the log-compression prior to compounding.

It should be understood that while FIGS. 3-4 are described separately, each processing system may be embodied in a single image processing system, such as image processing system 204. In this regard, utilization of image processing system 300 or image processing system 400 may be controlled via an operator (e.g., via user interface 211). For example, during one image acquisition and formation instance, the user may choose to use system 300. In another image acquisition and formation instance, the user may choose to use system 400. Advantageously, both systems 300, 400 may be embodied in a single image processing system such that a user or operator may selectively choose which system to use based on the application. In this regard, system 300 may be better suited for a particular situation than system 400, such that a user may use system 300 in that particular situation.

It should also be understood that in still further embodiments, a user may choose parts of systems 300 and 400 together. As such, the user may tailor the image processing system to their specific needs by advantageously identifying and selecting which components from system 300 and system 400 to use in the image formation process.

As such, as will be readily appreciated by those of skill in the art, the present disclosure is widely applicably with a high degree of configurability. While many examples are described in isolation, this description is meant for clarity and not meant to be limiting. Accordingly, many different implementation embodiments are contemplated by the present disclosure with all such embodiments intended to fall within the spirit and scope of the present disclosure.

It should be understood that the foregoing embodiments could be extended to other multiple firing harmonic frequency compounding combinations that can be generally expressed as follows. First, two or more ultrasound firings would be made. These firings would be divided into several groups. In this way, each group can share firings with other groups. For groups with more than one firing, the firings would be coherently combined to form tissue-generated harmonic signals. The harmonic signals can include sub-harmonic, ultra-harmonics, second order harmonic, and higher order harmonic. The number of groups with more than one firing could be one or more. The number of groups with one firing could also be one or more. For each group, the output of the coherent sum is detected in the case of two or more firings and the firing is directly detected in the case of one firing. All detected outputs are combined to form the compounded image as described above. The fundamental signals can be generated by a plurality of ways to combine the firings. The fundamental signals may also contain sub groups similar to the harmonic signals.

Although the figures show a specific order of method/system steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

Additionally, the format and symbols employed are provided to explain the logical steps of the schematic diagrams and are understood not to limit the scope of the methods/systems illustrated by the diagrams. Although various arrow types and line types may be employed in the schematic diagrams, they are understood not to limit the scope of the corresponding methods/systems. Indeed, some arrows or other connectors may be used to indicate only the logical flow of a method. For instance, an arrow may indicate a waiting or monitoring period of unspecified duration between enumerated steps of a depicted method. Additionally, the order in which a particular method occurs may or may not strictly adhere to the order of the corresponding steps shown. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in machine-readable medium for execution by various types of processors. An identified module of executable code may, for instance, comprise one or more physical or logical blocks of computer instructions, which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of computer readable program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in machine-readable medium (or computer-readable medium), the computer readable program code may be stored and/or propagated on in one or more computer readable medium(s).

The computer readable medium may be a tangible computer readable storage medium storing the computer readable program code. The computer readable storage medium may be, for example, but not limited to, an electronic, magnetic, optical, electromagnetic, infrared, holographic, micromechanical, or semiconductor system, apparatus, or device, or any suitable combination of the foregoing.

More specific examples of the computer readable medium may include but are not limited to a portable computer diskette, a hard disk, a random access memory (RAM), a read-only memory (ROM), an erasable programmable read-only memory (EPROM or Flash memory), a portable compact disc read-only memory (CD-ROM), a digital versatile disc (DVD), an optical storage device, a magnetic storage device, a holographic storage medium, a micromechanical storage device, or any suitable combination of the foregoing. In the context of this document, a computer readable storage medium may be any tangible medium that can contain, and/or store computer readable program code for use by and/or in connection with an instruction execution system, apparatus, or device.

The computer readable medium may also be a computer readable signal medium. A computer readable signal medium may include a propagated data signal with computer readable program code embodied therein, for example, in baseband or as part of a carrier wave. Such a propagated signal may take any of a variety of forms, including, but not limited to, electrical, electro-magnetic, magnetic, optical, or any suitable combination thereof. A computer readable signal medium may be any computer readable medium that is not a computer readable storage medium and that can communicate, propagate, or transport computer readable program code for use by or in connection with an instruction execution system, apparatus, or device. Computer readable program code embodied on a computer readable signal medium may be transmitted using any appropriate medium, including but not limited to wireless, wireline, optical fiber cable, Radio Frequency (RF), or the like, or any suitable combination of the foregoing.

In one embodiment, the computer readable medium may comprise a combination of one or more computer readable storage mediums and one or more computer readable signal mediums. For example, computer readable program code may be both propagated as an electro-magnetic signal through a fiber optic cable for execution by a processor and stored on RAM storage device for execution by the processor.

Computer readable program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The computer readable program code may execute entirely on the user's computer, partly on the user's computer, as a stand-alone computer-readable package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The program code may also be stored in a computer readable medium that can direct a computer, other programmable data processing apparatus, or other devices to function in a particular manner, such that the instructions stored in the computer readable medium produce an article of manufacture including instructions which implement the function/act specified in the schematic flowchart diagrams and/or schematic block diagrams block or blocks.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Accordingly, the present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

What is claimed:

1. An ultrasound machine, comprising:
an image acquisition device structured to acquire image data corresponding to an object of interest, wherein the image data includes channel data corresponding to each of at least three firings from the image acquisition device, and wherein the image data includes a fundamental component and a harmonic component;
an image processing system communicably coupled to the image acquisition device, the image processing system structured to isolate the fundamental component from the harmonic component by summing channel data from a set of firings in the at least three firings and subsequently combining the isolated fundamental and harmonic components; and
an image output device structured to provide an ultrasound image from the combined harmonic and fundamental components.

2. The ultrasound machine of claim 1, wherein the set of firings correspond to an identical amplitude and an identical phase.

3. The ultrasound machine of claim 1, wherein the channel data corresponding to the set of firings includes a higher transmission frequency relative to a transmission frequency for each of a remaining firing.

4. The ultrasound machine of claim 1, wherein the at least three firings is four firings, wherein a firing is inverted relative to a firing not in the set of firings.

5. The ultrasound machine of claim 4, wherein the channel data corresponding to one firing in the set of firings and the channel data corresponding to the firing that is inverted are stored in a memory device until channel data is obtained for the remaining two firings.

6. The ultrasound machine of claim 4, wherein the image processing system is structured to sum the inverted firings to isolate the harmonic component.

7. The ultrasound machine of claim 6, wherein the image processing system is structured to:
demodulate, using a quadrature demodulator, the harmonic component to a baseband frequency; and
filter, using a baseband filter, the demodulated harmonic component to remove non-harmonic frequency components.

8. The ultrasound machine of claim 7, wherein the quadrature demodulator is structured as a depth-dependent quadrature demodulator, and wherein the baseband filter is structured as a depth-dependent baseband filter such that removed non-harmonic frequency components changes as a function of penetration depth of the firing.

9. The ultrasound machine of claim 1, wherein the image processing system is structured to separately apply log compression on the isolated fundamental and harmonic components prior to combining the isolated fundamental and harmonic components.

10. The ultrasound machine of claim 1, wherein the image processing system is structured to:
combine channel data from two inverted firings to isolate the harmonic component;
combine channel data from one of the two inverted firings with channel data from a third firing to isolate the fundamental component; and
combine the isolated fundamental component and the isolated harmonic component incoherently.

11. An image processing system, comprising:
a beamformer module structured to receive channel data from each of at least three firings; and
a synthesis module communicably coupled to the beamformer module, the synthesis module structured to:
combine channel data corresponding to two inverted firings to isolate a harmonic component;
combine channel data from one of the two inverted firings with channel data from a third firing to isolate a fundamental component; and
combine the fundamental component with the harmonic component incoherently.

12. The image processing system of claim 11, wherein the synthesis module includes:
a quadrature demodulation module structured to separately demodulate the harmonic component and the fundamental component at a baseband frequency; and
a base-band filter structured to remove non-fundamental frequency components from the fundamental component and non-harmonic frequency components from the harmonic component.

13. The image processing system of claim 12, further comprising a log compression module, wherein the log compression module is structured to separately log compress the harmonic component and the fundamental component following the quadrature demodulation and the base-band filter, and wherein the separate log compression is before the fundamental and harmonic components are combined.

14. The image processing system of claim 12, wherein the quadrature demodulation is depth-dependent, wherein the demodulation varies as a function of depth of a firing.

15. The image processing system of claim 12, wherein the base-band filter is depth-dependent, wherein the filtering varies as a function of depth of a firing.

16. The image processing system of claim 11, further comprising a gain module, wherein the gain module is structured to:
apply an identical first weight to channel data from each of the two inverted firings to cancel the fundamental component and isolate the harmonic component when the channel data of two inverted firings are combined;
apply a second weight to the channel data from one of the two inverted firings; and
apply a third weight to the channel data from the third firing;
wherein the first weight is applied prior to combining the channel data from one of the inverted firings with channel data from the third firing to isolate the fundamental component.

17. The image processing system of claim 16, wherein the third weight is greater than the second weight.

18. The image processing system of claim 11, wherein the image processing system is used with ultrasonography system modes including A-mode, C-mode, Doppler mode, A-mode, harmonic mode, and acoustic radiation force imaging mode.

19. A method for reducing speckle in an ultrasound image, the method comprising:
receiving, by an image processing system, channel data specific to each of at least three firings from an image acquisition device;
combining, by the image processing system, channel data from two inverted firings to isolate a harmonic component;
combining, by the image processing system, channel data from one of the two inverted firings with channel data from a third firing to isolate a fundamental component;
log compressing, by the image processing system, each of the isolated harmonic and fundamental components separately; and
combining the log compressed isolated harmonic and fundamental components to form an image.

20. The method of claim 19, further comprising applying, by the image processing system, an identical first weight to the channel data from the two inverted firings prior to combining the two inverted firings.

21. The method of claim 19, further comprising applying, by the image processing system, a second weight to channel data from one of the two inverted firings, and a third weight to the channel data from the third firing prior to combining the channel data from one of the two inverted firings with channel data from the third firing.

22. The method of claim 21, wherein the third weight is greater than the second weight.

23. The method of claim 19, wherein each of the plurality of firings use Golay codes.
